(19) United States
(12) Patent Application Publication
(10) Pub. No.: US 2011/0046080 A1
(43) Pub. Date: Feb. 24, 2011

(54) Composition for Treating a Skin Disorder

(75) Inventor: Ali Rezai-Fard, Middlesex (GB)

Correspondence Address: MACMILLAN SOBANSKI & TODD, LLC, ONE MARITIME PLAZA FIFTH FLOOR, 720 WATER STREET, TOLEDO, OH, 43604-1619, US

(73) Assignee: Ali Rezai-Fard, Middlesex (GB)

(21) Appl. No.: 12/863,262

(22) PCT Filed: Jan. 16, 2009
(86) PCT No.: PCT/GB09/00113

§ 371 Date: Sep. 10, 2010

(30) Foreign Application Priority Data
Jan. 16, 2008 (GB) 0800791.6

(51) Int. Cl. A61K 31/704; A61K 31/01; A61P 17/00; A61P 17/06; A61P 17/10

(52) U.S. Cl. 514/26; 514/762

(57) ABSTRACT

The present invention relates to the use of a plant of the genus, Capsicum, or part thereof or extract thereof for treating or alleviating the symptoms of a skin condition or disorder, including dermatitis. The present invention also relates to compositions comprising the same for such use, together with methods of treating or alleviating the symptoms of eczema or dermatitis.

The present invention relates to a composition for treating a skin condition or disorder, in particular, Eczema or Dermatitis. More particularly, the present invention relates to the use of the whole, or part, of a plant of the genus Capsicum or an extract thereof, or compositions comprising same, in particular, pharmaceutical compositions, for treating or alleviating the symptoms of Dermatitis or Eczema. Other examples of skin conditions or disorders that can also be treated are psoriasis, acne, dandruff and Epidermolysis Bullosa (EB).

Psoriasis is a non-contagious disorder which affects the skin and joints. It commonly causes red scaly patches to appear on the skin. The scaly patches caused by psoriasis, called psoriatic plaques, are areas of inflammation and excessive skin production. Skin rapidly accumulates at these sites and takes on a silvery-white appearance.

The cause of psoriasis is not fully understood. There are two main hypotheses about the process that occurs in the development of the disease. The first considers psoriasis as primarily a disorder of excessive growth and reproduction of skin cells. The second hypothesis sees the disease as being an immune-mediated disorder in which the excessive reproduction of skin cells is secondary to factors produced by the immune system.

Acne vulgaris (commonly called acne) is a skin disease caused by changes in the pilsebaceous units (skin structures consisting of a hair follicle and its associated sebaceous gland). Severe acne is inflammatory but acne can also manifest in non-inflammatory forms. Acne lesions are commonly referred to as pimples, blemishes, spots, zits or acne.

Dandruff is a condition that is closely related to seborrhoeic eczema (and sometimes even referred to as seborrhoeic eczema). Dandruff is caused by excessive shedding of dead skin cells from the scalp and is an uninflamed form of seborrhoeic eczema.

Epidermolysis Bullosa (EB) is a rare genetic disease characterised by the presence of extremely fragile skin and recurrent blister formation, resulting from minor mechanical friction or trauma. There is also an acquired type of EB known as Epidermolysis Bullosa Acquisita, which is classified as an autoimmune disorder.

Dermatitis, a condition of the skin, derives its meaning from Greek and means inflammation of the skin. Eczema is a pruritic papulovesicular dermatitis occurring as a reaction to many endogenous and exogenous agents.

In the past, the term “dermatitis” was used to describe inflammation of the skin caused by factors outside the body, sometimes referred to as “contact or irritant” dermatitis, whereas “eczema” was used to describe inflammation of the skin that had no obvious external cause. However, at the present date, there is no longer believed to be a distinction between the respective terms in the art and they are now accepted to mean one and the same thing, and, as such, are used interchangeably. As a result, it is to be understood that the terms “dermatitis” and “eczema” utilised herein encompass the same conditions and are interchangeable.

Dermatitis or eczema affects both males and females equally. In general, about 10% of adults and 20% of children suffer from this disease. It typically starts before the age of five and can continue into adulthood. However, it is not uncommon for the disease to first show up later in life.

Although the underlying mechanism of eczema is not fully understood, it is categorised as an autoimmune disease. The causes of eczema are many and vary according to the particular form of the disease. In this connection, the three most common forms are atopic eczema, irritant eczema and allergic eczema.

Atopic eczema or dermatitis is an inherited condition and usually linked to asthma, allergic rhinitis and/or hay fever.

Irritant eczema, also known as irritant contact dermatitis, is understood to be caused by agents that strip the natural grease from the skin. Examples of such agents include soaps, detergents and various disinfectants.

Allergic eczema, also known as allergic contact dermatitis, is understood to be caused by an immune reaction to a substance which comes into contact with the skin. Common allergens include nickel, chromium, various plants, cosmetics and hair dyes.

Other forms of non-atopic dermatitis include infantile seborrhoeic dermatitis (cradle dermatitis), adult seborrhoeic dermatitis, varicose dermatitis and discoid dermatitis.

Dyshidrotic eczema, (also called pompholyx or vesicular eczema) usually affects the hands, although it may also develop on the feet. Small itchy bumps (“tapioca-like”) appear on the fingers. The bumps then develop into a rash. Dyshidrotic eczema is aggravated by physical or emotional stress, and runs in families.

Pompholyx eczema is an extremely itchy type of eczema associated with small to large blisters that affect the palms of the hands and/or the soles of the feet. It often occurs out of the blue, but it is also associated with atopic or contact eczema. Allergic contact eczema to nickel may appear as a pompholyx reaction (itchy blisters).

Napkin eczema is a form of irritant and/or allergic contact eczema that occurs in babies and young children, due to prolonged contact of the skin with urine resulting in contact eczema caused by ammonia, faeces and the effects of gut bacteria. Napkin eczema is a red rash easily diagnosed by its distribution in the nappy area and absence within the skin creases, where the nappy has not been in contact with the skin.

In mild forms, dermatitis is generally characterised by inflamed, irritated, red, dry, hot, scaling, lesions, swelling and almost always itching (pruritis) skin. In severe forms, the skin can become broken, and bleeding may follow. This may lead to bacterial and fungal infection. Furthermore, it is widely appreciated that the symptoms of the condition may be exacerbated by anxiety, stress and depression.

Some of the symptoms of dermatitis include the following conditions of the skin:

Lichenification: thick, leathery skin resulting from constant scratching and rubbing;

Papules: small raised bumps that may open when scratched. They may become crusty and infected;

Ichthyosis: dry, rectangular scales on the skin;

Keratosis pilaris: small, rough bumps, generally on the face, upper arms, and thighs;

Hyperlinear palms: increased number of skin creases on the palms;

Urticaria: hives (red, raised bumps). Arise often after exposure to an allergen, at the beginning of flares, or after exercise or a hot bath;

Cheilitis: inflammation of the skin on and around the lips;

Atopic pleat (Dennie-Morgan fold): an extra fold of skin that develops under the eye;

Hyperpigmented eyelids: eyelids that have become darker in colour from inflammation;

As will be appreciated, dermatitis can have severe implications on a patient's quality of life. For example, itching caused by the condition may disturb sleep patterns. In addition, it can also result in the need for special clothing and bedding that will not exacerbate or aggravate the condition.

With a view to treating the condition, it is known to use corticosteroid creams and ointments. One of the disadvantages associated with the use of such creams and ointments is that the base constituent of certain brands of known corticosteroid creams and ointments can act as an irritant. In addition, other known side effects of repeated or long-term use of topical corticosteroids include thinning of the skin, infection, pigmentation, growth suppression (in infants), and stretch marks on the skin. Furthermore, in infants, it is known that prolonged use of high-potency corticosteroid creams or ointments can result in adrenal suppression.

When topical treatments are not effective, it is also known to use systemic corticosteroids to treat the condition. Typically, these medications are used only in acute cases and are only administered for short periods of time. Disadvantageous side effects associated with using systemic corticosteroids include skin damage, thinned or weakened bones, high blood pressure, high blood sugar and cataract. In addition, high doses have been associated with avascular necrosis of the femoral head. Moreover, such treatments have been linked to mental disturbances including paranoia and depression. Furthermore, high doses of corticosteroids may cause Cushing's syndrome, with moon face, striae and acne.

It is also known to use antihistamines to treat dermatitis; however, they are often sedating and anticholinergic.

For unusually widespread, recalcitrant, or disabling cases, experimental treatments such as immunomodulatory and immunosuppressive agents such as cyclosporin, tacrolimus, interferon gamma and phototherapy have also been used. However, it has been documented that these can compromise the immune system.

In addition to conventional treatments, some herbal remedies, mainly of Chinese origin, have been found to be beneficial. However, they too suffer from drawbacks. For example, the majority of such remedies have been found to contain high concentrations of steroids that are harmful to the body, whilst others have powerful immunosuppressant properties, or contain dangerous toxins. The use of these preparations is further complicated by the fact that their efficacy has not been reliably demonstrated by rigorous clinical trials.

As will be appreciated there is a need to provide new products, compositions or agents which can treat or alleviate the symptoms of skin conditions and disorders and in particular, dermatitis or eczema, which at least addresses some of the problems associated with the known products, compositions or agents identified above.

Considering the nature of the condition, and the discomfort often associated with applying topical medicaments regularly to often sore skin, it is the particular object of the present invention to provide an effective orally dosed treatment for these skin conditions.

It is also the object of the present invention to provide a new product, composition or agents that can also be used to treat or alleviate the symptoms of other related skin conditions such as psoriasis, acne, dandruff and Epidermolysis Bullosa (EB) It is an object of the present invention to provide such a product, composition or agent.

### SUMMARY OF INVENTION

In a first aspect of the present invention there is provided the use of a plant from the genus, Capsicum, or a part thereof or an extract therefrom for the manufacture of a medicament for use in treating or alleviating the symptoms of a skin condition, wherein the medicament is suitable for oral, nasal or rectal administration.

In a second aspect of the present invention there is provided the use of at least one seed of a plant of the genus, Capsicum, or an extract thereof for the manufacture of a medicament for use in treating or alleviating the symptoms of a skin condition, wherein the medicament is suitable for oral, nasal or rectal administration.

In a third aspect of the present invention is there provided the use of a plant from the genus, Capsicum, or a part thereof or an extract therefrom for the manufacture of a medicament for use in treating or alleviating the symptoms of a skin condition, wherein the medicament is substantially free of capsaicin or other capsaicinoids.

In a fourth aspect of the present invention there is provided the use of at least one seed of a plant of the genus, Capsicum, or an extract thereof for the manufacture of a medicament for use in treating or alleviating the symptoms of a skin condition, wherein the medicament is substantially free of capsaicin or other capsaicinoids

In a fifth aspect of the present invention, there is provided the use of a saponin of the genus Capsicum, or an analogue, mimetic or derivative thereof for the manufacture of a medicament for use in treating or alleviating the symptoms of a skin condition, wherein the medicament is suitable for oral, nasal or rectal administration.

In a sixth aspect of the present invention, there is provided the use of a saponin extracted from a plant of the genus, Capsicum, or an analogue, mimetic or derivative thereof for the manufacture of a medicament for use in treating or alleviating the symptoms of a skin condition wherein the medicament is substantially free of capsaicin or other capsaicinoids

In a seventh aspect of the present invention, there is provided the use of a triterpene or triterpenoid extracted from a plant of the genus, Capsicum, or an analogue, mimetic or derivative thereof for the manufacture of a medicament for use in treating or alleviating the symptoms of a skin condition medicament is substantially free of capsaicin or other capsaicinoids.

In an eighth aspect of the present invention, there is provided the use of a triterpene or triterpenoid of the genus Capsicum, or an analogue, mimetic or derivative thereof for the manufacture of a medicament for use in treating or alleviating the symptoms of a skin condition, wherein the medicament is suitable for oral, nasal or rectal administration.

In a ninth aspect of the present invention, there is provided a composition comprising at least one seed of a plant of the genus Capsicum suitable for use in treating or alleviating the symptoms of a skin condition, wherein in the medicament is suitable for oral, nasal or rectal administration.

In a tenth aspect of the present invention, there is provided a composition comprising at least one seed of a plant of the genus Capsicum suitable for use in treating or alleviating the symptoms of a skin condition, wherein in the medicament is substantially free of capsaicin or other capsaicinoids.

In an eleventh aspect of the present invention there is provided a pharmaceutical composition comprising an extract of a plant of the genus Capsicum and at least one pharmaceutical acceptable excipient suitable for use in treating or alleviating the symptoms of skin condition wherein the medicament is suitable for oral, nasal or rectal administration.

In a twelfth aspect of the present invention there is provided a pharmaceutical composition comprising an extract of a plant of the genus Capsicum and at least one pharmaceutical acceptable excipient suitable for use in treating or alleviating the symptoms of a skin condition wherein the medicament is substantially free of capsaicin or other capsaicinoids.

### DETAILED DESCRIPTION OF THE INVENTION

Plants of the genus, Capsicum have edible capsular fruits containing many seeds. Members of this genus include cayenne, habanero, jalapeno, paprika and tabasco chilli peppers. This genus is a member of the Solanaceae family, such family also including tomato, tobacco, and petunia. Plants of the genus, in particular, their fruits, are primarily used as a food source; although certain members of the genus, in particular extracts thereof, have been used for medicinal purposes. For example, capsaicin, found in the placenta of chilli peppers, namely, the white fibrous material that holds the seeds, has been used to treat pain.

WO 93/23061 discloses the use of topical medicaments comprising plant extracts from pungent botanicals, (including Capsicums) primarily to treat skin conditions caused by microorganisms and in particular fungal infections. Examples are given in this document demonstrating the effectiveness of such topical formulations to treat athletes foot and ring worm. The document suggests that these topical formulations will be effective in treating many other skin conditions, including eczema and dandruff. This document also teaches that the therapeutic effect lies in the pungent components of these plants, the capsaicinoids, and in particular capsaicin.

Capsaicin is the primary active ingredient of the pungent taste of the Capsicum plant genus. Other capsaicinoid compounds that are found in this plant genus are dihydrocapsaicin, nordihydrocapsaicin, homodihydrocapsaicin, homocapsaicin and nonivamide. Capsaicin is the main capsaicinoid present in the Capsicum plants, typically corresponding to 70% by weight of the capsaicinoids present.

WO 93/23061 discloses only topical treatments of the Capsicum containing compositions and systemic administration via injection. The document does not disclose oral administration, teaching instead that digestive decomposition prevents therapeutic activity of these botanical containing compositions.

The inventor, a long-term sufferer of eczema, has surprisingly discovered that by eating chilli peppers (belonging to C. annuum and C. frutescens) the symptoms of his eczema are eradicated. The preferred chilli peppers used were green cayenne chilli peppers (C. annuum) and green and red bird's eye chilli peppers (belonging to both C. annuum and C. frutescens). Moreover, the inventor has discovered that the therapeutic effect is not provided by capsaicin present in the chilli peppers.

The chilli peppers used in the present invention have purchased in normal supermarkets and food shops. They are sold under labels such as; thin chillies, thin Indian chillies, Thai chillies and finger chillies. The colour of the chillies is not important to the present invention.

There are only a few common species of chilli peppers (although there are many cultivars). Examples of these species are:

Capsicum annuum—includes many common varieties such as paprika, cayenne, and jalapeños

Capsicum frutescens—includes the tabasco peppers and Thai chillies

Capsicum chinense—includes the hottest peppers such as the naga, habanero, Datil and Scotch bonnet

Capsicum pubescens—includes the South American rocoto peppers

Capsicum baccatum—includes the South American aji peppers

To aid with an understanding of the invention, an account of the inventor's observations and investigations giving rise to the arrival at the present invention will be described hereinbelow. It is to be understood that the following account should in no way be construed as limiting as to the scope of protection conferred by the present application.

The inventor of the present invention has been suffering from dermatitis or eczema since childhood. Areas of the inventor's body affected by the disease or condition include the auxiliary regions, fingers, face, eyelids and soles of his feet.

In general, the inventor has found topical steroid preparations to be largely unsatisfactory and the benefits temporary.

By sheer chance, the inventor noticed that on eating chilli peppers, of the Capsicum genus of plant, his symptoms caused by the condition disappeared. By way of further explanation, the inventor found this natural product to be equally effective on each and every subsequent occasion it has been used by the inventor. This has been used for about the last 8 years. Moreover, the inventor has not used any other medication, since discovering the benefit of consuming this product, because it has been particularly effective in treating his condition. For each episode that involved the consumption of chilli peppers for a few weeks, a large batch of chilli peppers was obtained beforehand and stored in the refrigerator.

In order to confirm that there was a link between the inventor eating chilli peppers and the symptoms of his eczema being eradicated, the inventor ate chocolate and other food products, which were known to him to exacerbate the symptoms of his condition.

Once the symptoms of his eczema had returned, he embarked on an administration regimen involving the consumption of 5 to 10 fruits of chilli peppers twice a day, of typical size and shape. He noticed that within about 14 days of starting such a treatment his symptoms had almost completely disappeared and that within 21 days of starting such a treatment the symptoms were eradicated. He also noticed that by carrying on with the consumption of 5 to 10 fruits of chilli peppers twice per day for 3 month resulted in his symptoms being eradicated for up to 12 months.

Once the symptoms reappeared, the inventor observed that a second course of treatment was found to be equally effective.

As the inventor had only been carrying out tests in relation to the pungent forms of the members of the Capsicum family, he conducted further investigations to establish whether the agent responsible for the beneficial effect was in some way related to the constituent making them pungent, namely, capsaicin. In this connection, although the literature indicated that capsaicin was not naturally present within the seeds, it could, with time, contaminate the surface of the seeds. As a result, the seeds of the chilli pepper were first removed and separated from the rest of the berry or fruit or pod manually i.e. to minimise any contamination of the seeds by capsaicin. The seeds were then treated to remove any capsaicin that may be present on the seed's surface. To this end, as capsaicin is almost insoluble in cold water, but soluble in warm and hot water, the seeds were thoroughly washed with “hot-to-the-touch” (approximately 50° C.) water repeatedly. The seeds were qualitatively checked for the presence of the pungent capsaicin by taste and found to be absent.

To treat the seeds to remove any trace of capsaicin and other capsaicinoids, the temperature of the water used to wash the seeds may be from 30 to 100° C., preferably from 40 to 80° C. and most preferably from 50 to 60° C.

The amount of seed to be ingested was then standardised to approximately 400 to 500 seeds and taken with food twice a day. Within 21 days of starting the treatment regimen the inventor's symptoms of dermatitis that had appeared on his fingers, eyelids and face had cleared completely.

To confirm this finding, and after taking the necessary action to bring back his symptoms, the above process was repeated. Once again, within 21 days, the inventor's symptoms had cleared completely.

Therefore, and based on the inventor's investigations to date, it would appear that the agent or constituent responsible or causative of the observed beneficial effect is located or found in the seeds of the chilli pepper.

And furthermore therefore the ingested seeds, extracts and compositions of the present invention do not require the presence of capsaicinoids for their therapeutic activity. The medicaments and compositions of the present invention may be entirely free, or at least substantially free of capsaicin and other capsaicinoids.

By substantially free of capsaicin or other capsaicinoids we mean that the levels of these compounds in the compositions and medicaments are at or below about the level that can be achieved by washing the seeds in warm water.

Preferably the medicaments or compositions of the present invention comprise less than 0.5%, preferably less than 0.1%, preferably less than 0.05% preferably less than 0.01%, preferably less than 0.005% and most preferably less than 0.001% by weight of capsaicinoids in the plant or seed extract used to prepare the medicament.

If possible there are undetectable levels of capsaicinoid compounds in the medicaments or compositions of the present invention, when analysed using normal chemical analytical techniques, such as HPLC or GC.

Storage

The inventor also noticed during his investigations that the beneficial effect of the seeds was more likely to be present in active concentration if the chilli peppers or their seeds were stored prior to their ingestion in a cool environment of between 0 and 10 degrees Centigrade. This storage gave beneficial results after only 48 hours.

However the effect of storage at low temperature was improved if it was maintained for between 2 days and 26 weeks, preferably between 3 days and 16 weeks, more preferably between 4 days and 12 weeks, more preferably between 5 days and 8 weeks, most preferably between 1 week and 3 weeks.

Therefore, and based on the inventor's investigations to date, it would appear that the agent or constituent responsible or causative of the observed beneficial effect is located or found at the requisite levels i.e. at a high enough concentration in the seeds of the chilli pepper when the fruit or seed is stored between 0 and 10 degrees Centigrade for at least 2 days but most preferably for 1 to 3 weeks.

Although not wishing to be bound by any theory as to which constituent present within the seeds may be responsible for treating the inventor's condition, it is possible that it is one of the saponins, known as capsicidins, which are known to be present within the seeds of plants of the genus Capsicum.

It is also possible that the constituent present within the seeds of plants of the genus Capsicum, responsible for treating the inventor's condition, is a triterpene or triterpenoid.

As will be appreciated, it is to be understood that the products of the present invention can be administered topically, enterally including orally, intranasal, and rectally, Parenterally (injection and infusion) and other systemic methods. In this connection, as the production of such formulations is well known in the art, there is believed to be no need to provide any further information about the preparation of such formulations herein.

Within the context of the present application, the following terms utilised above and below are to be attributed the meanings provided hereinbelow:

Capsicum is to be understood as encompassing any plant species of the genus Capsicum. Generally, members of the group have edible capsular fruits containing many seeds. Members include, but are not limited to, C. annuum, C. frutescens, C. chinense, C. pendulum, C. pubescens, C. minimum, C. baccatum, C. abbreviatum, C. anomalum, C. breviflorum, C. buforum, C. brasilianum, C. campylopodium, C. cardenasii, C. chacoense, C. ciliare, C. ciliatum, C. chlorocladium, C. coccineum, C. cordiforme, C. cornutum, C. dimorphum, C. dusenii, C. exile, C. eximium, C. fasciculatum, C. fastigiatum, C. flexuosum, C. galapagoensis, C. geminifolum, C. hookerianum, C. lanceolatum, C. leptopodum, C. luteum, C. microcarpum, C. minutiflorum, C. mirabile, C. parvifolium, C. praetermissum, C. schottianum, C. scolnikianum, C. stramonifolium, C. tetragonum, C. tovarii, C. villosum, C. violaceum. It is also to be understood that the present invention encompasses the use of the whole or any part of a plant of the genus Capsicum, for example, its fruit, seed(s), leaves or root; any constituent, derivative or extract obtained therefrom, as well as any synthetic version of any constituent, derivative or extract obtained from such plant or part thereof.

It is also to be understood that saponin, a glycoside, encompasses not only neutral saponins, which are derivatives of steroids, including steroidal glycoside saponins and steroidal glycoalkaloid saponins, but also, acid saponins including triterpene saponins and triterpenoid saponins. It is also to be understood that such term includes, but not limited to, the group of saponins known as capsicidins (also known as capsicidines) including capsicosides A-G. It is also to be understood to include, but not limited to, solasodine, solanidine and solanine and furostanol type saponins. It is also to be understood that the present invention encompasses the use of saponins which are extracted from a plant or part thereof of the genus, Capsicum, or saponins which have been produced synthetically. It is also to be understood that the present invention also encompasses the use of analogues, mimetics and derivatives of saponins of the genus, Capsicum.

It is also to be understood that the present invention encompasses the use of triterpenes or triterpenoids which are extracted from a plant or part thereof of the genus, Capsicum, or triterpenes or triterpenoids which have been produced synthetically. It is also to be understood that the present invention also encompasses the use of analogues, mimetics and derivatives of triterpenes triterpenoids of the genus, Capsicum.

It is also to be understood that in addition to saponins, triterpenes and triterpenoids, other extracts from a seed of a plant of the genus Capsicum which may be responsible for the beneficial effect include, but are not limited to, 24-(R)-ethyl-lophenol, 24-methylene-cycloartanol, 24-methyl-lanost-9(11)-en-3-β-ol, 24-methyl-lophenol, 31-norcycloartanol, 31 -nor-lanost-8-en-3-β-ol, 31-nor-lanost-9(11)-en-3-β-ol, 31-nor-lanosterol, 4-α-14-α-24-trimethyl-cholesta-8(24)-dien-3-β-ol, 4-α-24-dimethyl-cholesta-7,24-dien-3-β-ol, 4-α-methyl-5-α-cholest-8(14)-en-3-β-ol, β-amyrin, citrostadienol, cycloartanol, cycloartenol, cycloeucalenol, gramisterol, lanost-8-en-3-β-ol, lanosterol, lophenol, lupeol and obtusifoliol.

It is also to be understood that the present invention also relates to methods for treating or alleviating the symptoms of dermatitis or eczema, the method comprising administering any plant of the genus, Capsicum, or part thereof or extract thereof, or composition comprising same, to a subject, in particular, a human subject. Preferably, such administration is effected orally.

Extraction of the Active

Extraction of the active agent from the plant of the genus, Capsicum, or part thereof, (in particular from the seeds) can be carried out by any method known in the art. For example, solvent extraction of the crushed seeds with a range of different organic solvents or an aqueous solution could yield the active compound. Any compounds extracted into these solutions can be isolated and identified using standard laboratory techniques, such as HPLC. Further testing of these isolated compounds will enable the identification of the active species.

Once identified, the compounds can also then be prepared synthetically. This can be achieved using standard organic synthetic techniques well known in the art.

Pharmaceutical compositions of the present invention can be prepared from both the natural extracts and synthetically derived equivalents.

Dosing

The fruit, seed or active ingredient from the plant of the genus Capsicum seeds are suitably administered in an effective amount to alleviate or prevent the symptoms of dermatitis or eczema. Suitable dosages are in the range of from about 0.1 seeds to about 30 seeds per kg of body weight per day, for example from about 1 seed to about 20 seeds per kg of body weight per day (or equivalent amounts of the whole fruit or of the active ingredients extracted from the seeds).

Effective dosages of the fruits of capsicums to treat the skin conditions are therefore between 1 to 100, preferably 2 to 75, preferably 3 to 50, preferably 5 to 30 and most preferably between 10 to 20 fruits of capsicums per dose.

Effective dosages of the seeds of Capsicums to treat the skin conditions are therefore between 10 to 2000, preferably 50 to 1750, preferably 100 to 1000, preferably 200 to 750 and most preferably between 400 to 500 seeds of Capsicums per dose.

Dosing may be between 1 and 8 times per day. More preferably, between 2 to 4 times per day. Dosing can be carried out at meal times, with regular food intake, or it can be carried out in the absence of other foods.

Dosing may be carried out for as long as the skin condition requires. This may require days, weeks or even months of dosing. The dosing may cease once the symptoms have cleared. The dosing may need to be resumed if symptoms re-emerge at a later date. In some cases the dosing may need to be continual to maintain a symptom free condition.

Pharmaceutical Compositions

A pharmaceutical composition can be prepared to treat skin disorders, comprising a plant extract from a plant of the genus Capsicum and a pharmaceutically acceptable excipient. Excipients may comprise one or more antiadherents, binders, coatings, disintegrants, fillers/diluents, flavours, colourants, glidants, lubricants, preservatives, sorbents and sweeteners.

Preferably the pharmaceutical composition is prepared using an extract from the seeds of a plant of the genus Capsicum. The active may be found to be more than one compound. The active may be a saponin, triterpene, triterpenoid or a derivative thereof.

The extract may be the individual active (or group of actives) isolated from the plant material. This can be achieved by any known chemical extraction method known in the art.

Preferably the active is extracted from the seeds of a Capsicum plant. Alternatively the composition may be derived from using simply the crushed form of the entire seeds.

The extract, once identified can be prepared synthetically for use in the pharmaceutical compositions. The extract may also comprise analogues, mimetics and pharmaceutically acceptable derivatives of the active extract.

The pharmaceutical composition can be delivered to the patient in the form of a tablet, capsule, aerosol, gas, enema, suppository. liquid solution or powder. The pharmaceutical composition of the present invention can be delivered to the body of the patient in any way known in the art. This includes inhalation, oral, parenteral, vaginal and rectal dosage forms.

Most preferably however the pharmaceutical composition will be delivered to the body orally and take the form of a capsule or tablet.

The person skilled in the art of pharmaceutical formulations will be able to provide alternative formulations to those in the Examples and that are suitable for other methods of (nasal, etc) administration.

If formed from the active ingredient, the pharmaceutical composition will comprise a an amount of active per dose equivalent to the weight of active indicated for the treatment comprising the fruits and seeds above.

Alternatively the dose of the active ingredient may be between 1 and 500 mgs, preferably between 5 and 400 mgs, preferably between 10 and 300 mgs, preferably between 25 and 250 mgs, preferably between 50 and 200 mgs and more preferably between 100 and 150 mgs of the active ingredient per dose.

Treating Further Conditions

Eczema is considered to be an autoimmune disease. Autoimmune diseases arise from an overactive immune response of the body against substances and tissues normally present in the body. In other words, the body attacks its own cells.

Given the effectiveness of medicaments of the present invention that are based on extracts from the Capsicum fruit, and particular the seeds, in treating eczema and dermatitis, these treatments and compositions potentially also provide therapeutic benefit to sufferers of other autoimmune diseases.

Examples of other autoimmune diseases are acute disseminated encephalomyelitis (ADEM), Addison's disease, alopeccia areata, antiphospholipid antibody syndrome (APS), autoimmune haemolytic anemia, autoimmune hepatitis, bullous pemphigoid, coeliac disease, Crohn's disease, Goodpasture's syndrome, Hashimoto's disease, idiopathic thrombocytopenic purpura, multiple sclerosis, pernicious anaemia, rheumatoid arthritis and ulcerativecolitis.

Accordingly the Inventor considers that plants from the genus, Capsicum, or a part thereof or an extract therefrom can be used for the manufacture of a medicament to treat or alleviate the symptoms of autoimmune diseases, including in particular but not limited to, those listed above.

Preferably the parts of the plants, if used, are the seeds and the extracts, if used, are taken from the seeds. Preferably the medicament is suitable for oral administration.

One of the main symptoms of eczema and dermatitis is intense itching. The medicaments of the present invention alleviate the symptoms of itching that are caused by eczema, dermatitis and psoriasis.

The inventor considers the medicaments and compositions of the present invention would be effective treatments of other medical conditions that cause itching. Examples of other conditions include allergic reaction, chickenpox, fungal infections, insect bites and insect stings.

Therefore plants from the genus, Capsicum, or a part thereof or an extract therefrom can be used in the manufacture of a medicament to treat or alleviate the symptoms of itching, wherein the itching may have been caused by allergic reaction, chickenpox, fungal infections, insect bites and insect stings.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

The following Examples illustrate the invention.

### EXAMPLE 1

Tablet

5-15 weight % Capsicum plant extract, in the form of the isolated active compounds, or crushed seeds, or powdered seeds.

Up to 70 weight % of a filler, such as lactose powder, microcrystalline cellulose, starch, sucrose, or a mixture of thereof.

Up to 25 weight % of optional ingredients, such as disintegrants, colourants, lubricants, and flavourings.

The tablet may also possess a coating. This can be to improve stability, disguise the flavour or control location of absorption in the body.

### EXAMPLES 2 AND 3

Capsules

A gelatin capsule can be filled with whole seeds from a Capsicum plant or from a. powder formed from the ground seeds.

Alternatively a gelatin capsule maybe provided with a liquid solution (a “softgel” formulation) of the active extract in a suitable liquid excipient environment, such as water and glycerin.

1. A medicament comprising at least one seed of a plant of the genus, Capsicum, or an extract thereof, for the treatment of, or alleviating the symptoms of, a skin condition, wherein the medicament is suitable for oral, nasal or rectal administration, wherein the medicament is substantially free of capsaicin or other capsaicinoids, and wherein the skin condition comprises one or more of the conditions selected from psoriasis, acne, dandruff, epidermolysis bullosa and dermatitis.

2. The medicament of according to claim 1, wherein the plant of the genus Capsicum is selected from the group consisting of C. annuum, C. frutescens, C. chinense, C. pendulum, C. pubescens, C. minimum, C. baccatum, C. abbreviatum, C. anomalum, C. breviflorum, C. buforum, C. brasilianum, C. campylopodium, C. cardenasii, C. chacoense, C. ciliare, C. ciliatum, C. chlorocladium, C. coccineum, C. cordiforme, C. cornutum, C. dimorphum, C. dusenii, C. exile, C. eximium, C. fasciculatum, C. fastigiatum, C. flexuosum, C. galapagoensis, C. geminifolum, C. hookerianum, C. lanceolatum, C. leptopodum, C. luteum, C. microcarpum, C. minutiflorum, C. mirabile, C. parvifolium, C. praetermissum, C. schottianum, C. scolnikianum, C. stramonifolium, C. tetragonum, C. tovarii, C. villosum, C. violaceum and their hybrids.

3. The medicament according to claim 2, wherein the plant is C. annuum.

4. The medicament according to claim 2, wherein the plant is C. frutescens.

5. The medicament according to claim 1, wherein the extract comprises a saponin.

6. The medicament according to claim 5, wherein the saponin is a capsicidine selected from the group consisting of capsicoside A, capsicoside B, capsicoside C, capsicoside D, capsicoside E, capsicoside F, capsicoside G or solasodine, solanidine and solanine.

7. The medicament according to claim 1 wherein the extract comprises a triterpene or triterpenoid.

8. A composition comprising at least one seed of a plant of the genus, Capsicum suitable for use in treating or alleviating the symptoms of a skin condition, wherein the medicament is suitable for oral, nasal or rectal administration, wherein the medicament is substantially free of capsaicin or other capsaicinoids, and wherein the skin condition comprises one or more of the conditions selected from, psoriasis, acne, dandruff and epidermolysis bullosa and dermatitis.

9. A pharmaceutical composition suitable for treating a skin condition, comprising an extract of, one or more seeds of a plant of the genus Capsicum and at least one pharmaceutically acceptable excipient wherein the medicament is suitable for oral nasal or rectal administration, wherein the medicament is substantially free of capsaicin or other capsaicinoids and wherein the skin condition comprises one or more of the conditions selected from, psoriasis, acne, dandruff epidermolysis bullosa and dermatitis.

10. The pharmaceutical composition of claim 9 wherein the seed extract comprises a saponin, or triterpene or a triterpenoid, or a combination thereof.

11. The composition of claim 8, wherein the plant of the genus Capsicum is selected from the group consisting of C. annuum, C. frutescens, C. chinense, C. pendulum, C. pubescens, C. minimum, C. baccatum, C. abbreviatum, C. anomalum, C. breviflorum, C. buforum, C. brasilianum, C. campylopodium, C. cardenasii, C. chacoense, C. ciliare, C. ciliatum, C. chlorocladium, C. coccineum, C. cordiforme, C. cornutum, C. dimorphum, C. dusenii, C. exile, C. eximium, C. fasciculatum, C. fastigiatum, C. flexuosum, C. galapagoensis, C. geminifolum, C. hookerianum, C. lanceolatum, C. leptopodum, C. luteum, C. microcarpum, C. minutiflorum, C. mirabile, C. parvifolium, C. praetermissum, C. schottianum, C. scolnikianum, C. stramonifolium, C. tetragonum, C. tovarii, C. villosum, C. violaceum and their hybrids.

12. The composition of claim 8, wherein the plant is C. annuum.

13. The composition of claim 8, wherein the plant is C. frutescens.

14. The composition of claim 12 wherein the plant variety is cayenne chilli peppers.

15. The composition of claim 12, wherein the plant variety is bird's eye chilli peppers.

16. The medicament according to claim 1, wherein the composition or medicament comprises the fruit or seed of the genus Capsicum, that has been stored between 0 and 10 degrees Centigrade, for at least 2 days but preferably for 1 to 3 weeks.

17. The medicament according to claim 1, wherein the seeds have been treated to substantially remove capsaicin or other capsaicinoids.

18. The medicament according to claim 17 wherein the treatment to the seeds comprises washing the seeds in warm water with a temperature of between 40-80° C.

19. The composition of claim 9, wherein the plant of the genus Capsicum is selected from the group consisting of C. annuum, C. frutescens, C. chinense, C. pendulum, C. pubescens, C. minimum, C. baccatum, C. abbreviatum, C. anomalum, C. breviflorum, C. buforum, C. brasilianum, C. campylopodium, C. cardenasii, C. chacoense, C. ciliare, C. ciliatum, C. chlorocladium, C. coccineum, C. cordiforme, C. cornutum, C. dimorphum, C. dusenii, C. exile, C. eximium, C. fasciculatum, C. fastigiatum, C. flexuosum, C. galapagoensis, C. geminifolum, C. hookerianum, C. lanceolatum, C. leptopodum, C. luteum, C. microcarpum, C. minutiflorum, C. mirabile, C. parvifolium, C. praetermissum, C. schottianum, C. scolnikianum, C. stramonifolium, C. tetragonum, C. tovarii, C. villosum, C. violaceum and their hybrids.

20. The composition of claim 9, wherein the plant is C. annuum.

21. The composition of claim 9, wherein the plant is C. frutescens.

22. The composition of claim 20 wherein the plant variety is cayenne chilli peppers.

23. The composition of claim 21, wherein the plant variety is bird's eye chilli peppers.

24. The composition of claim 10, wherein the plant of the genus Capsicum is selected from the group consisting of C. annuum, C. frutescens, C. chinense, C. pendulum, C. pubescens, C. minimum, C. baccatum, C. abbreviatum, C. anomalum, C. breviflorum, C. buforum, C. brasilianum, C. campylopodium, C. cardenasii, C. chacoense, C. ciliare, C. ciliatum, C. chlorocladium, C. coccineum, C. cordiforme, C. cornutum, C. dimorphum, C. dusenii, C. exile, C. eximium, C. fasciculatum, C. fastigiatum, C. flexuosum, C. galapagoensis, C. geminifolum, C. hookerianum, C. lanceolatum, C. leptopodum, C. luteum, C. microcarpum, C. minutiflorum, C. mirabile, C. parvifolium, C. praetermissum, C. schottianum, C. scolnikianum, C. stramonifolium, C. tetragonum, C. tovarii, C. villosum, C. violaceum and their hybrids.

25. The composition of claim 10, wherein the plant is C. annuum.

26. The composition of claim 10, wherein the plant is C. frutescens.

27. The composition of claim 25, wherein the plant variety is cayenne chilli peppers.

28. The composition of claim 25, wherein the plant variety is bird's eye chilli peppers.

29. The composition according to claim 8, wherein the composition or medicament comprises the fruit or seed of the genus Capsicum, that has been stored between 0 and 10 degrees Centigrade, for at least 2 days but preferably for 1 to 3 weeks.

30. The composition according to claim 8, wherein the seeds have been treated to substantially remove capsaicin or other capsaicinoids.

31. The composition according to claim 30, wherein the treatment to the seeds comprises washing the seeds in warm water with a temperature of between 40-80° C.

32. The composition according to claim 9, wherein the composition or medicament comprises the fruit or seed of the genus Capsicum, that has been stored between 0 and 10 degrees Centigrade, for at least 2 days but preferably for 1 to 3 weeks.

33. The composition according to claim 9, wherein the seeds have been treated to substantially remove capsaicin or other capsaicinoids.

34. The composition according to claim 33, wherein the treatment to the seeds comprises washing the seeds in warm water with a temperature of between 40-80° C.
